(19) United States
(12) Patent Application Publication
(10) Pub. No.: US 2012/0089203 A1
(43) Pub. Date: Apr. 12, 2012

(54) ELECTRICAL CONNECTIONS FOR USE IN IMPLANTABLE MEDICAL DEVICES

(75) Inventor: Ryan Shaffer, Carrollton, TX (US)

(73) Assignee: ADVANCED NEUROMODULATION SYSTEMS, INC., Plano, TX (US)

(21) Appl. No.: 13/237,476

(22) Filed: Sep. 20, 2011

Related U.S. Application Data
(60) Provisional application No. 61/392,319, filed on Oct. 12, 2010.

(51) Int. Cl. A61N 1/375

(52) U.S. Cl. 607/72; 607/2

(57) ABSTRACT

There is disclosed various embodiments of an electrical connection for electrically connecting implantable medical devices together, such as an electrical connector for connecting an implantable pulse generator to a medical lead. In one embodiment, the electrical connection may include a body having a longitudinal opening defined therein for receiving an electrode of the medical lead and a spring coupled to the body and positioned within the opening, the spring having a curved surface for engaging the surface of the electrode.

### TECHNICAL FIELD

The present application is generally related to electrical connections for use in medical devices, specifically for electrical connections for an implantable medical device system within a patient.

### BACKGROUND INFORMATION

Neurostimulation therapy is frequently associated with patients having a wide variety of diseases and disorders. In general, neurostimulation therapy works by applying an electrical current to the nerves which may be causing symptoms, such as chronic pain.

In neuromodulation systems, such as spinal cord stimulation systems (“SCS”), a thin wire or lead with electrodes at its distal end is implanted into a patient in the location to be treated, such as within the epidural space of the patient to deliver the electrical pulses to the spinal neural tissue. A pulse generator is electrically connected to the proximal end of the electrical lead with the pulse generator typically implanted within a subcutaneous pocket within the patient. The pulse generator generates electrical pulses or current which stimulates the nerves around the electrodes at the treatment location.

The efficacy of the electrical stimulation in facilitating the management of pain of the patient depends upon applying the electrical pulses to the appropriate neural tissue. The connection between the pulse generator and the leads should be sufficiently tight to make a good electrical connection to allow the transmission of electrical signals between the pulse generator and the leads. If the electrical connection between the pulse generator and the leads are loose, the signal will not be transmitted, and the effectiveness of the electrical stimulation may be greatly reduced.

### SUMMARY

There is disclosed various embodiments of an electrical connection for electrically connecting implantable medical devices together, such as an electrical connector for connecting an implantable pulse generator to a medical lead. In one embodiment, the electrical connection may include a body having a longitudinal opening defined therein for receiving a portion of an electrode of the medical lead and a spring coupled to the body and positioned within the opening, the spring having a curved surface for engaging the surface of the electrode.

The foregoing has outlined rather broadly certain features and/or technical advantages in order that the detailed description that follows may be better understood. Additional features and/or advantages will be described hereinafter which form the subject of the claims. It should be appreciated by those skilled in the art that the conception and specific embodiment disclosed may be readily utilized as a basis for modifying or designing other structures for carrying out the same purposes. It should also be realized by those skilled in the art that such equivalent constructions do not depart from the spirit and scope of the appended claims. The novel features, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the appended claims.

### BRIEF DESCRIPTION OF THE DRAWINGS

FIG. 1 is a perspective view of one embodiment of a stimulation system which may use certain aspects of the present invention.

FIG. 2 is a perspective view of one embodiment of an implantable lead which may be used in the stimulation system of FIG. 1.

FIG. 3a is an exploded perspective view of a stimulation device showing a header housing component removed.

FIG. 3b is a detailed exploded perspective view of a stimulation device showing additional header components removed.

FIG. 4a is a perspective view of one embodiment of an electrical connector which may be incorporated into the system of FIG. 1.

FIG. 4b is a different perspective view of the electrical connector of FIG. 4a.

FIG. 4c is a perspective view of a component of the electrical connector of FIG. 4a.

FIG. 4d is a perspective view of a component of the electrical connector of FIG. 4a.

FIG. 4e is a front elevation view of the electrical connector of FIG. 4a.

FIG. 5 is a front elevation view of an alternative embodiment of an electrical connector.

FIG. 6a is a front elevation view of an alternative embodiment of an electrical connector.

FIG. 6b is a front elevation view of an alternative embodiment of an electrical connector.

FIG. 7a is a front elevation view of an alternative embodiment of an electrical connector.

FIG. 7b is a front elevation view of an alternative embodiment of an electrical connector.

FIG. 8 is a front elevation view of an alternative embodiment of an electrical connector.

FIG. 9 is a front elevation view of an alternative embodiment of an electrical connector.

FIG. 10 is a front elevation view of an alternative embodiment of an electrical connector.

FIG. 11a is a section view of an alternative embodiment of an electrical connector.

FIG. 11b is a front elevation view of an alternative embodiment of the electrical connector of FIG. 11a.

FIG. 12 is a perspective view of one embodiment of a medical device which may employ certain aspects of the present invention.

FIG. 13a is a perspective view of the embodiment of FIG. 12 with certain components removed for clarity.

FIG. 13b is a perspective view of the embodiment of FIG. 12 with certain components removed for clarity.

### DETAILED DESCRIPTION

For the purposes of promoting an understanding of the principles of the present invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

When directions, such as upper, lower, top, bottom, clockwise, counter-clockwise, are discussed in this disclosure, such directions are meant to only supply reference directions for the illustrated figures and for orientations of components in the figures. The directions should not be read to imply actual directions used in any resulting invention or actual use. Under no circumstances, should such directions be read to limit or impart any meaning into the claims.

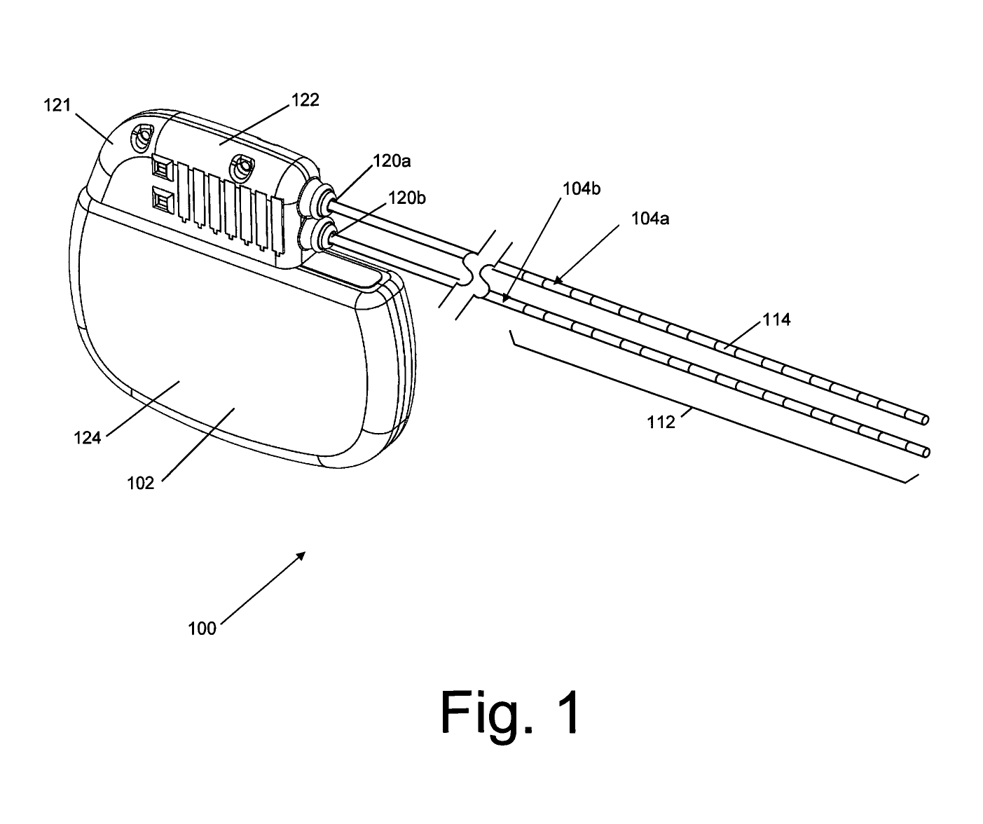

An exemplary neurostimulation system which may employ certain aspects of the present invention is illustrated in FIG. 1. A neurostimulation system 100 includes a stimulation source, such as an implantable pulse generator 102 (“IPG”) which may be coupled to one or more stimulation leads 104a and 104b. As will be explained below, the IPG 102 typically includes a power source (such as a battery) and electronics (such as hardware, software, or embedded logic components) for generating electrical stimulation signals or pulses.

In this example, the system 100 employs the two stimulation leads 104a and 104b, but any number of stimulation leads could be employed and are within the scope of the present invention. Each of the leads 104a and 104b may generally be configured to transmit one or more electrical signals from the IPG 102 to a spinal nerve, a peripheral nerve, or other tissue. As illustrated in FIG. 1 by the break lines, the leads 104a and 104b are not meant to accurately represent the actual length of the leads relative to the pulse generator.

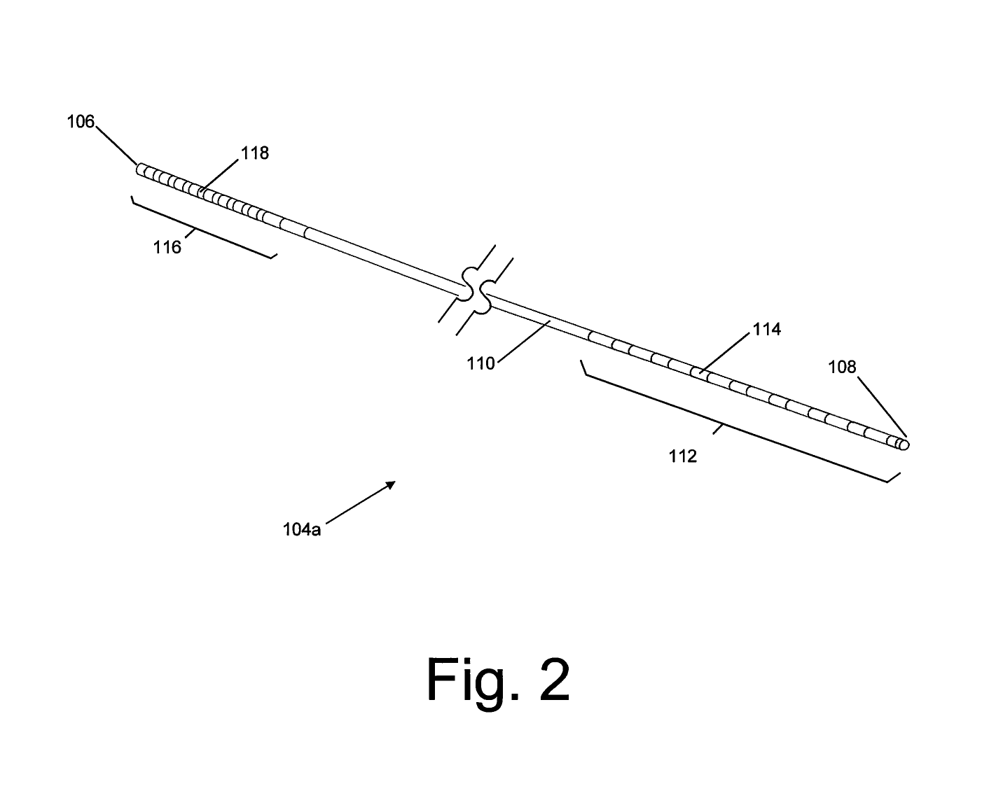

FIG. 2 is a partial isometric illustration of a portion of the lead 104a (or the lead 104b). (Again the lead 104a is cut in length by break lines so that details of the leads may be clearly visible.) The lead 104a includes a proximal end 106 and a distal end 108. The lead 100 further comprises a flexible lead body 110 that extends from proximal end 106 to the distal end 108. In certain embodiments, one or more lumens (not shown) may extend through the lead body 110 and may be used for housing one or more stiffeners (not shown).

In certain embodiments, the lead body 110 may be a structure having a round or substantially round cross-section. Alternatively, the cross-section of the lead body 110 may be configured in any number of cross-sectional shapes appropriate for a specific application in which the lead will be used. Depending on the particular application, the diameter of the lead body may be any size, though a smaller size is more desirable for lead applications such as neurological and myocardial mapping/ablation and neuromodulation and stimulation.

The lead body 110 may be formed of an extrusion or insulating material typically selected based upon biocompatibility, biostability and durability for the particular application. The insulator material may be silicone, polyurethane, polyethylene, polyamide, polyvinylchloride, PTFT, EFTE, or other suitable materials known to those skilled in the art. Alloys or blends of these materials may also be formulated to help control the relative flexibility, torqueability, and pushability of lead 104a. In certain embodiments, the insulative material of lead body 110 may be substantially composed of a compliant PURSIL® or CARBOSIL® silicone-urethane copolymer material. In some applications, compliant material characteristic enables the lead body 110 to elongate significant amounts at relatively low stretching forces.

Adjacent to the distal end 108 of lead 104a is a stimulation electrode region 112 comprising, in this embodiment, a plurality of eight stimulation electrodes 114. Adjacent to proximal end 106 of lead 104a is a connector region 116 that, in this embodiment, also comprises a plurality of eight connector or terminal electrodes 118 which are sized to couple with the IPG 102 (as illustrated in FIG. 1). For purposes of illustration only, the lead 104a is shown with eight stimulation electrodes 114 and eight connector electrodes 118. As will be appreciated by those skilled in the art, any number of conductors and electrodes may be utilized as desired to form lead 104a. Generally, embodiments have the same number of stimulation electrodes as connector electrodes.

In certain embodiments, both the plurality of stimulation electrodes 114 and the plurality of connector electrodes 118 may be formed of biocompatible, conductive materials such as stainless steel, platinum, gold, silver, platinum-iridium, stainless steel, MS35N, or other conductive materials, metals or alloys known to those skilled in the art. In some embodiments, as illustrated in FIG. 2, the plurality of stimulation electrodes 114 and the plurality of connector electrodes 118 may be ring or cylindrical electrodes which encircle portions of the stimulation electrode region 112 and connector region 116, respectively. Other types, configurations and shapes of electrodes as known to those skilled in the art may be used with embodiments.

One or more conductors (not shown) extend along a substantial portion of the lead body 110 to electrically connect the connector or terminal electrodes 118 to the corresponding stimulation electrodes 114. The conductors of the lead 104a may be maintained in electrical isolation by the insulative material of the lead body 110.

In certain embodiments, the conductors may be formed of a conductive material having desirable characteristics such as biocompatibility, corrosion resistance, flexibility, strength, low resistance, etc. The conductors may take the form of solid wires, drawn-filled-tube (DFT), drawn-brazed-strand (DBS), stranded wires or cables, ribbon conductors, or other forms known or recognized to those skilled in the art. The composition of the conductors may include aluminum, stainless steel, MP35N, platinum, gold, silver, copper, vanadium, alloys, or other conductive materials or metals known to those of ordinary skill in the art. In some embodiments, the number, size, and composition of the conductors will depend on the particular application for the lead, as well as the number of electrodes. An example of a commercially available stimulation lead is the Octrode™ lead available from St. Jude Medical.

Turning back to FIG. 1, the leads 104a and the 104b are illustrated connected to the IPG 102 via receptacles 120a and 120b defined within a header 121, respectively. The connector electrodes 118 are not visible in FIG. 1 because they are positioned within a header housing 122 of the header 121. However, the plurality of stimulation electrodes 114 are visible at the distal ends of the leads 104a and 104b.

The IPG 102 may use a housing 124 to enclose circuitry (not shown) for generating the electrical pulses for application to neural tissue of the patient. The circuitry enclosed in the IPG housing 124 may include one or more microprocessors or other circuitry including pulse generating circuitry, control circuitry, communication circuitry, recharging circuitry, and a battery or power source for the device. An example of pulse generating circuitry is described in U.S. Patent Publication No. 20060170486 entitled “PULSE GENERATOR HAVING AN EFFICIENT FRACTIONAL VOLTAGE CONVERTER AND METHOD OF USE,” which is incorporated herein by reference. A microprocessor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling with an external charging device is described in U.S. Pat. No. 7,212,110, entitled “IMPLANTABLE DEVICE AND SYSTEM FOR WIRELESS COMMUNICATION,” which is incorporated herein by reference. An example of a commercially available IPG 102 is the EON™ pulse generator available from St. Jude Medical.

The IPG 102 is usually implanted within a subcutaneous pocket created under the skin by a physician. The leads 104a and 104b are typically mechanically and electrically coupled to the pulse generator 102 and thus may be used to conduct the electrical pulses from the implant site of the pulse generator 102 to the targeted nerve tissue via a plurality of stimulation electrodes 114. For example, the stimulation electrode region 112 of leads 104a and 104b may be positioned within the epidural space of the patient to deliver electrical stimulation to spinal nerves to treat chronic pain of the patient.

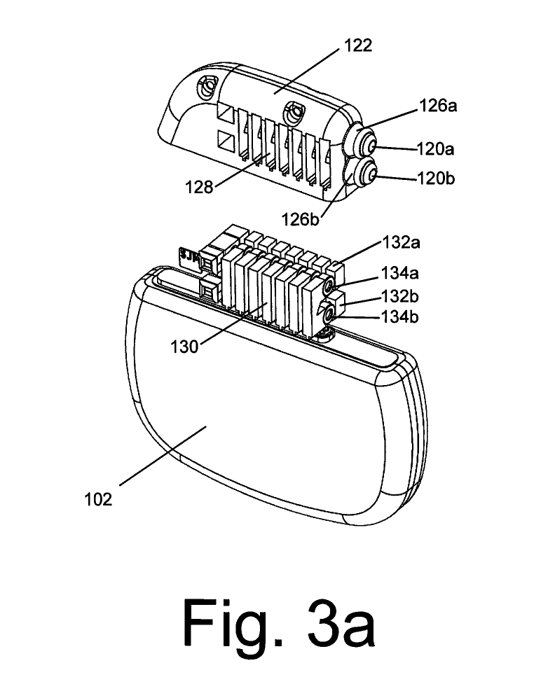

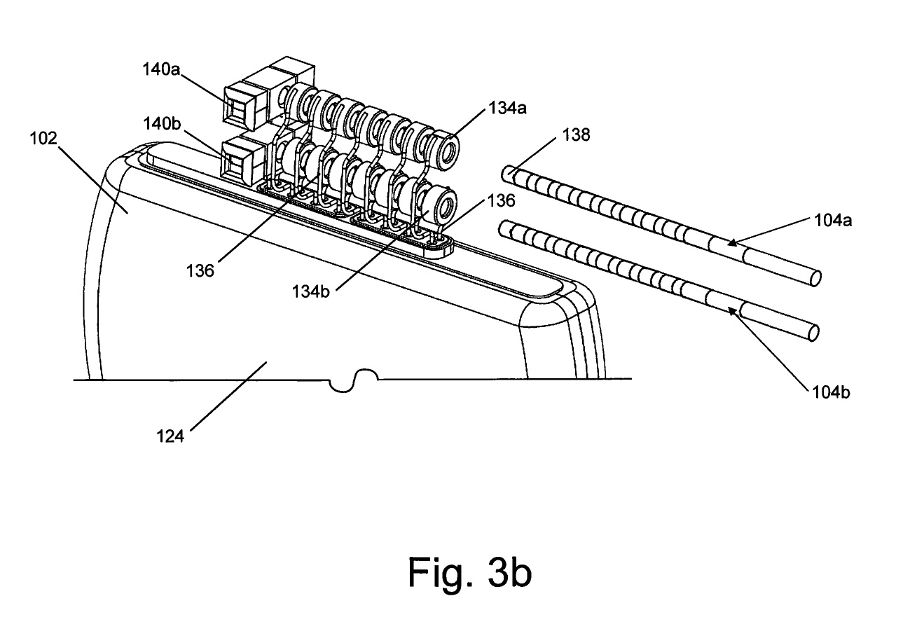

FIG. 3a is a partially exploded view of the pulse generator 102 with a header housing 122 positioned above the IPG 102 for clarity. In certain embodiments, the header housing 122 is made from a relatively compliant material using silicone-based materials and may be formed using injection molding techniques. However, if a sharp object used during the implantation procedure were to contact a compliant material, the compliant material could be punctured somewhat easily. The puncture could allow entry of body fluids and cause the patient to experience electrical stimulation in the subcutaneous implantation pocket. Consequently, in other embodiments, the header housing 122 may be made from a relatively high durometer polymer such as polyetheretherketone (“PEEK”) or Bionate® polycarbonate urethane. In yet other embodiments, the header housing 122 may incorporate an outer layer made from a relatively compliant material (such as silicone-based materials) and an inner protective layer made from a relatively high durometer polymer layer such as PEEK or Bionate® to protect the inner components of the header components. In yet other embodiments, a high durometer polyermer layer may be formed on the exterior of the header housing to act as a shield to protect the inner portion of the housing which is made from a relatively compliant material.

In certain embodiments, the receptacles 120a and 120b may incorporate strain relief elements 126a and 126b to assist in protecting the leads 104a and 104b from higher bending stresses which may be induced at the receptacles 120a and 120b. The strain relief elements 126a and 126b may also assist in providing a seal between the receptacles 120a and 120b and the electrodes 104a and 104b. As illustrated in FIG. 3a, the receptacles 120a and 120b are configured in an above-below manner. However, in other embodiments the receptacles 120a and 120b may be configured to receive the stimulation leads 104a and 104b in a side-by-side manner. Alternatively, any number of lead receptacles could be used with the header and pulse generator.

Defined within side walls of the header housing 122 is a plurality of recesses 128 for housing a first plurality of fillers 130. In the illustrative embodiment, two pluralities of opposing fillers 132a and 132b are housed in recesses (not shown) on the opposing side of the header housing 122.

The combination of fillers 130 and opposing fillers 132a and 132b position and hold two pluralities of electrical connectors 134a and 134b. The fillers 130, 132a and 132b are made from a compliant material. Once the fillers 130, 132a and 132b are assembled and positioned within the header housing 122, the compliant material characteristic of the fillers holds electrical connectors 134a and 134b in place by applying an elastomeric force to the electrical connectors. Additionally, when the header 121 is fully assembled and stimulation leads are placed in the header 121 through strain relief elements 126a and 126b, the various conductive elements are sealed within the components of the header 121. Specifically, when implantable pulse generator 102 is implanted within a patient, the housings and strain relief ports are designed to seal and prevent the electrical components from contacting bodily fluids.

FIG. 3b is a partial perspective view of certain header components of the pulse generator 102 with the header housing 122 and the fillers 130, 132a and 132b removed for clarity. The proximal ends of the leads 104a and 104b are also illustrated showing them linearly aligned with the plurality of electrical connectors 134a and 134b, respectively. The electrical connectors 134a and 134b are longitudinally spaced apart to match the longitudinal spacing of the connector electrodes 118 of the leads 104a and 104b so that when the ends of the leads 104a and 104b are correctly positioned into the plurality of electrical connectors 134a and 134b, the electrodes will be in contact with the electrical connectors.

A plurality of feedthrough wires 136 extend through the IPG housing 124 of the pulse generator 102 to electrically couple each of the connectors in the plurality of connectors 134a and 134b to pulse generation circuitry positioned within IPG housing 124. In certain embodiments, the feedthrough wires 136 are welded or soldered to an exterior surface of each connector in the plurality of connectors 134a and 134b.

As will be explained in detail later, each connector in the plurality of the connectors 134a and 134b has a bore or opening sized to accommodate a connector electrode 118 of the leads 104a and 104b. Thus, when the leads 104a and 104b are inserted into the plurality of connectors 134a and 134b, an electrical connection can be established between the plurality of connector electrodes 118 and the interior circuitry of the pulse generator 102.

In certain embodiments, there may also be a “dummy” electrode 138 positioned at the distal end portion of the connector region 116 of the lead 104a or 104b. In such embodiments, the dummy electrode 138 may not be electrically coupled to a conductor, but is provided as a structural support to aid in coupling the leads 104a and 104b to the IPG 102. A set screw (not shown) may be used to secure the dummy electrode 138 to the header 121. Self-sealing access ports 140a and 140b allow for access to these set screws (not shown) so they can be turned to secure the electrode 138 to the header 121.

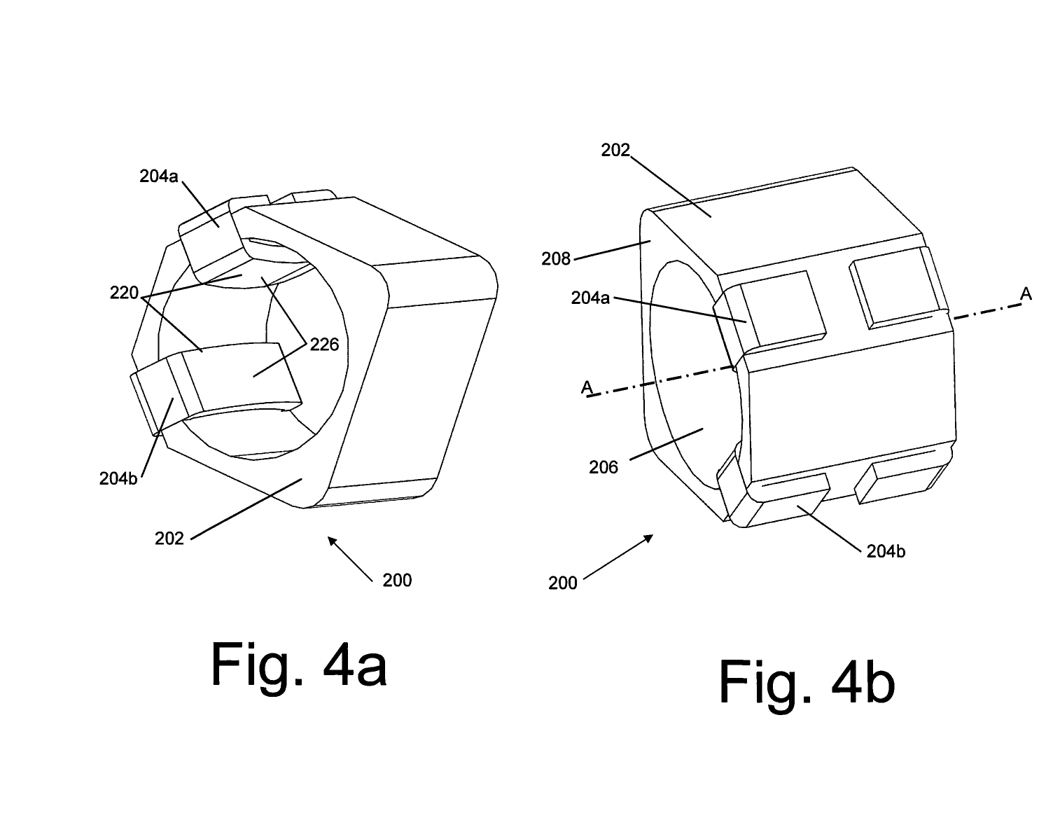

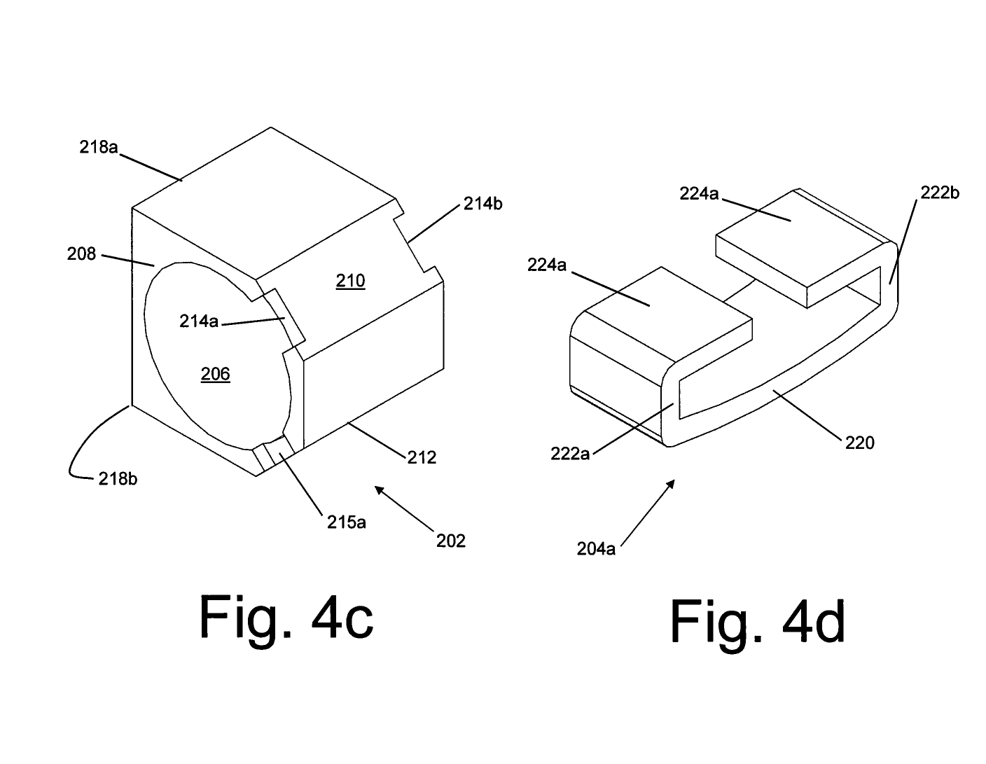

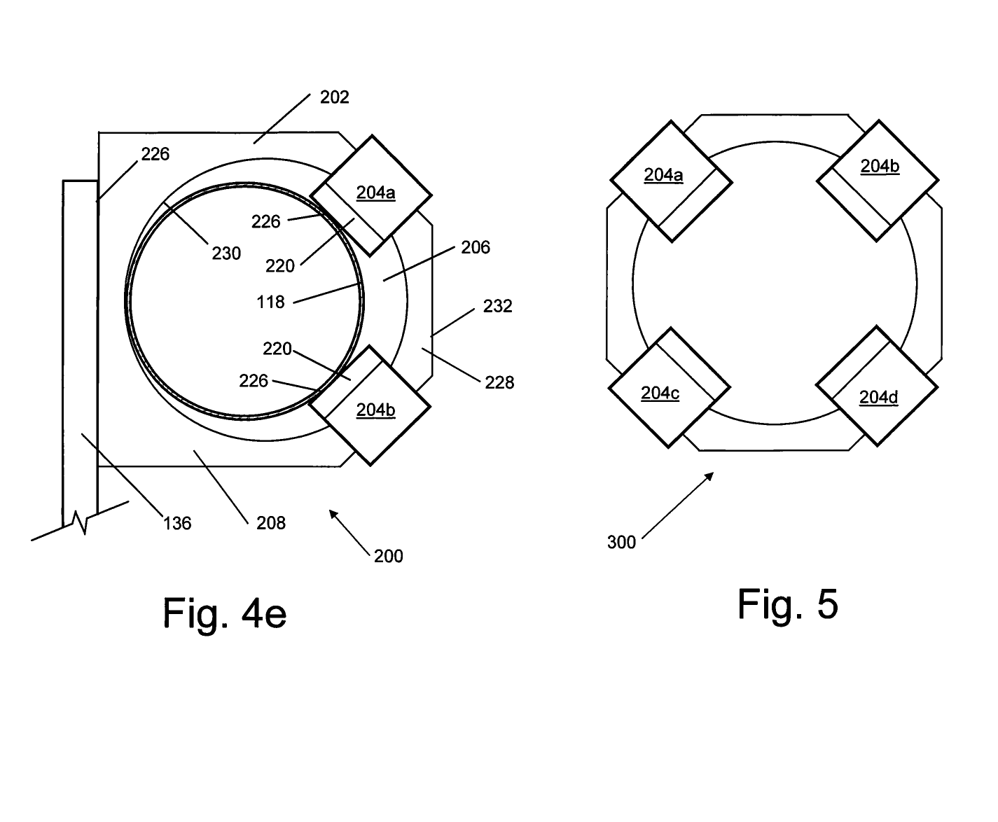

FIG. 4a depicts one embodiment of an electrical connector 200 from a first view which may be used with the system 100 described above in the previous figures. The electrical connector 200 is one embodiment of an electrical connector which could be used as one of the plurality of connectors 134a or 134b described above. FIG. 4b depicts the electrical connector 200 from another view. In the illustrated embodiment, the electrical connector 200 comprises a housing 202, a spring 204a and a spring 204b.

FIG. 4c is a perspective view of the housing 202 with the springs 204a and 204b removed for clarity. In certain embodiments, the housing 202 is generally square or rectangular in cross-sectional shape and may be formed from a rectangular bar or block. In the illustrative embodiment, there is a longitudinal axis A-A (FIG. 4b) and a longitudinal bore 206 about the axis A-A running from one transverse edge or face 208 to the opposing transverse edge or face (not shown). In this embodiment, two corners of the housing 202 have been chamfered down to form a side surface 210 and a chamfered side surface 212. Notches 214a and 214b are defined at the opposing edges of the chamfered side surfaces 210. Similar notches (only notch 215a is visible in FIG. 4c) are defined on the opposing ends of the side surface 212. In some embodiments, corner edges 218a and 218b may have a slight chamfering to reduce sharp edges as illustrated in FIG. 4a.

In one embodiment, the housing 202 may be machined or formed from a conductive material. To reduce an occurrence of oxidation, corrosion or both on the connector 200, the housing 202 may be formed or machined from bars of platinum, platinum-iridium, a platinum alloy or another conductive material resistant to corrosion and/or oxidation. Because such material is relatively expensive, alternative embodiments could be formed or machined from the appropriate biocompatible, conductive materials such as stainless steel, gold, silver, MS35N, or other conductive materials, metals or alloys known to those skilled in the art. Other embodiments of the housing 202 may be made from an appropriate conductive material, such as stainless steel or MP35N and be plated with platinum or a platinum-iridium alloy. In yet, other embodiments, the housing 202 may be made from a non-conductive material.

FIG. 4d is a perspective view of a spring, for instance spring 204a, with the housing 202 removed. The finished spring 204a comprises a curved portion 220 having transverse arm portions 222a and 222b extending from the curved portion 220. At a predetermined distance, the transverse arm portions 222a and 222b are bent towards each other to form longitudinal arm portions 224a and 224b. The spring 204a may be made from any type of the conductive materials described above in reference to the housing 202.

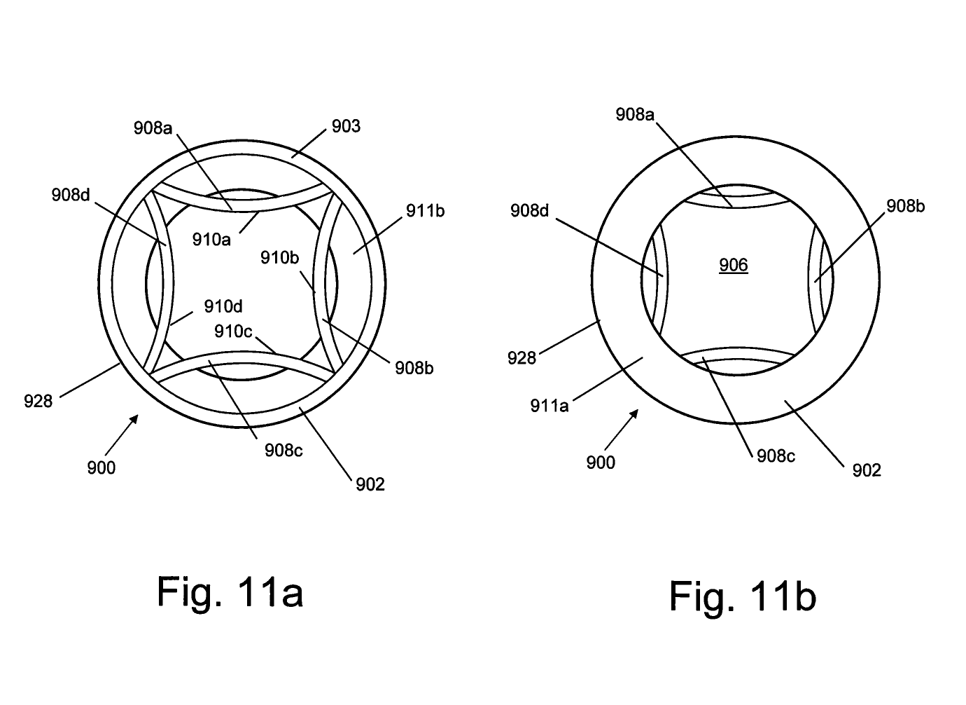

In order to assemble certain embodiments of the connector 200, a strip of conductive material (not shown) is longitudinally positioned within the bore 206 and next to an interior surface adjacent to the notches 214a and 214b (FIG. 4c). For purposes of this patent specification, the term “longitudinally positioned” means the length of the strip is generally placed parallel to the longitudinal axis of the bore 206. In contrast, the term “radially positioned” means the length of a strip is generally placed radially around the longitudinal axis of the bore 206 (as illustrated in FIG. 11a). During assembly of certain embodiments, the strip may be longitudinally positioned so that roughly the same length of the strip extends out from both faces of the housing 202 at the notches 214a and 214b. The strip may then be bent to form transverse arms 222a and 222b which fit within the notches 214a and 214b, respectively. Thus, in certain embodiments, the notches 214a and 214b help position the strip relative to the housing 202.

In certain embodiments, the curved portion 220 of the strip may bow towards the center of the bore 206 due to the bending of the strip forming a convex surface. Portions of the transverse arms may then be bent towards each other to form longitudinal arm portions 224a and 224b as illustrated in FIG. 4b and FIG. 4d to form the spring, for instance the spring 204a. The spring 204a may then be tacked welded to the housing 202. The spring 204b (FIG. 4a) may be bent and coupled to housing 202 is a similar manner to form the assembled electrical connector 200 as illustrated in FIG. 4a.

When the springs 204a and 204b are coupled to the housing 202, the curved portion 220 will bow out towards the center of the bore 206, but the curved portion is thin enough to allow a predetermined amount of flexing when pressure is applied to the curved or convex surface 226.

FIG. 4e is a front view of the connector 200 showing the bore 206, the transverse face 208 and the springs 204a and 204b coupled to the housing 202. When a connector electrode 118 (from a lead 104a or 104b) is inserted into the bore 206, the curved portion 220 of the springs 204a and 204b will yield, but be biased towards their manufactured or assembled shape. This biasing creates a pressure on the connector electrode 118 which forms a tight connection between an exterior surface of the connector electrode and the curved surface 226 of the springs 204a and 204b. In some embodiments where the housing 202 is also conductive, a tight electrical connection will also form between the connector electrode 118 and an interior surface 230 of the bore 206.

In embodiments where the material of the housing 202 is conductive, a feedthrough wire 136 may be welded to an exterior surface 226 as illustrated in FIG. 4e. Thus, an electrical connection can be established from the feedthrough wire 136 to a connector electrode 118 via the housing 202 and the springs 204a and 204b.

In other embodiments where the housing 202 is not conductive, the feedthrough wire 136 may wrap around the housing 202 on the opposing side 232 so that the wire can be welded directly to the springs 204a and 204b. An electrical connection would then be established from the feedthrough wire 136 to the connector electrode 118 via the springs 204a and 204b. Other electrical connectors in the pluralities of electrical connectors 134a and 134b (FIG. 3b) could be coupled to a corresponding feedthrough wire of the plurality of feedthrough wires 136 in a similar manner.

FIG. 5 illustrates an alternative embodiment of a connector 300 using four springs 204a-204d. The connector 300 may be similar to the embodiments of connector 200 discussed above except that all four corners have chamfers and notches to hold the four springs 204a-204d. Alternative embodiments may include any number of springs, from a single spring to four or more springs.

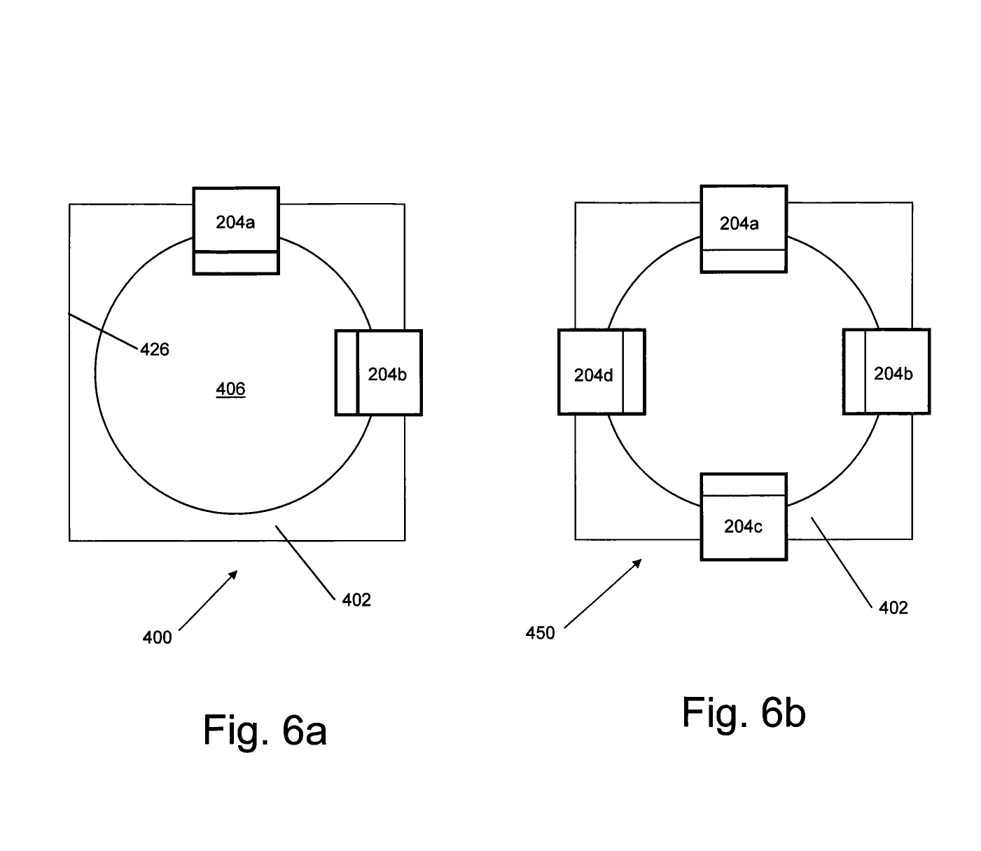

FIG. 6a illustrates an alternative embodiment of the connector 400. In this embodiment, the corners are not chamfered to any significant extent. The springs 204a-204b are not attached at the chamfered corners, but are attached at the mid-section of each side of the housing 402. In certain embodiments, notches (not shown), similar to notches 214a and 214b described in reference to FIG. 4a above, may be defined in the side walls of the housing 402 to help position the springs during assembly. In embodiments where the material of the housing 402 is conductive, a feedthrough wire (not shown) may be welded to an exterior surface 426. Thus, an electrical connection can be established from the feedthrough wire (not shown) to a connector electrode positioned within the bore 406 via the housing 402 and the springs 204a and 204b.

FIG. 6b illustrates an alternative embodiment for a connector 450. The connector 450 is similar to the connector 400 except that four springs 204a-204d are coupled to the housing 402.

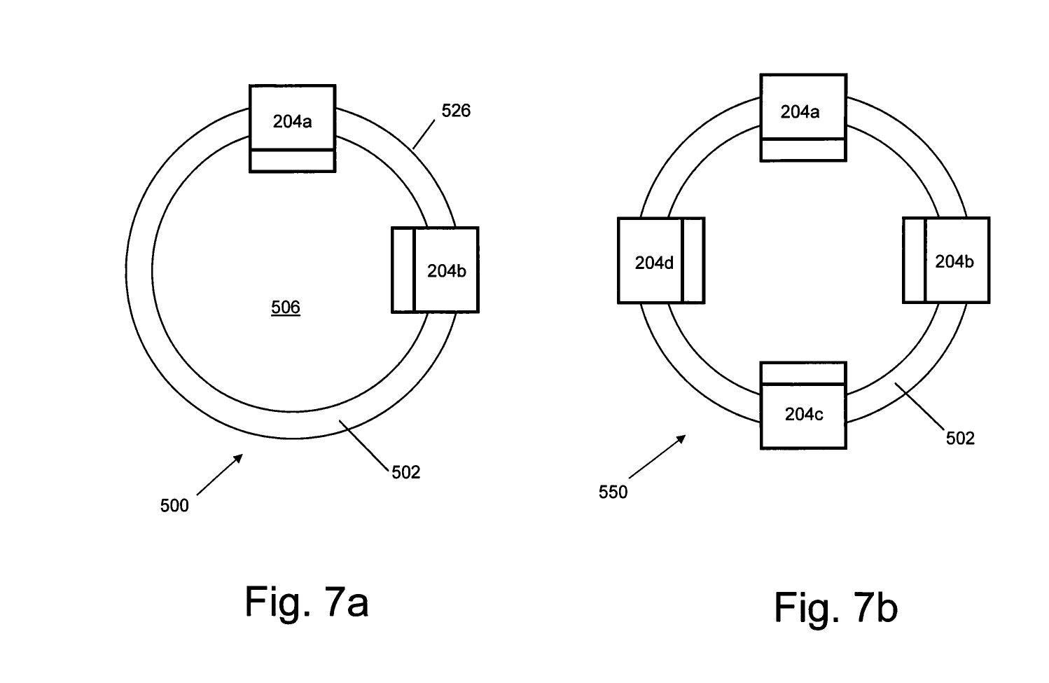

FIG. 7a illustrates an alternative embodiment of a connector 500. In this embodiment, the housing 502 is formed from a tube rather than a block with a longitudinal bore. In the illustrative embodiment, the springs 204a-204b are attached at two quarter points of the circular section of the tube. In certain embodiments, notches (not shown) similar to notches 214a and 214b described in reference to FIG. 4a above, may be defined at the ends of the tubular housing 502 to help position the springs 204a-204b during assembly. In embodiments where the material of the housing 502 is conductive, a feedthrough wire (not shown) may be welded to an exterior surface 526. Thus, an electrical connection can be established from the feedthrough wire (not shown) to a connector electrode when the connector electrode is positioned within the bore 506 via the housing 502 and the springs 204a and 204b.

FIG. 7b illustrates an alternative embodiment of a connector 550. The connector 550 is similar to the connector 500 except that four springs 204a-204d are coupled to the housing 502. Although the embodiments illustrated in FIGS. 7a and 7b illustrate the use of two and four springs, respectively, it is contemplated that a single spring, three springs, or more that fours springs could be utilized.

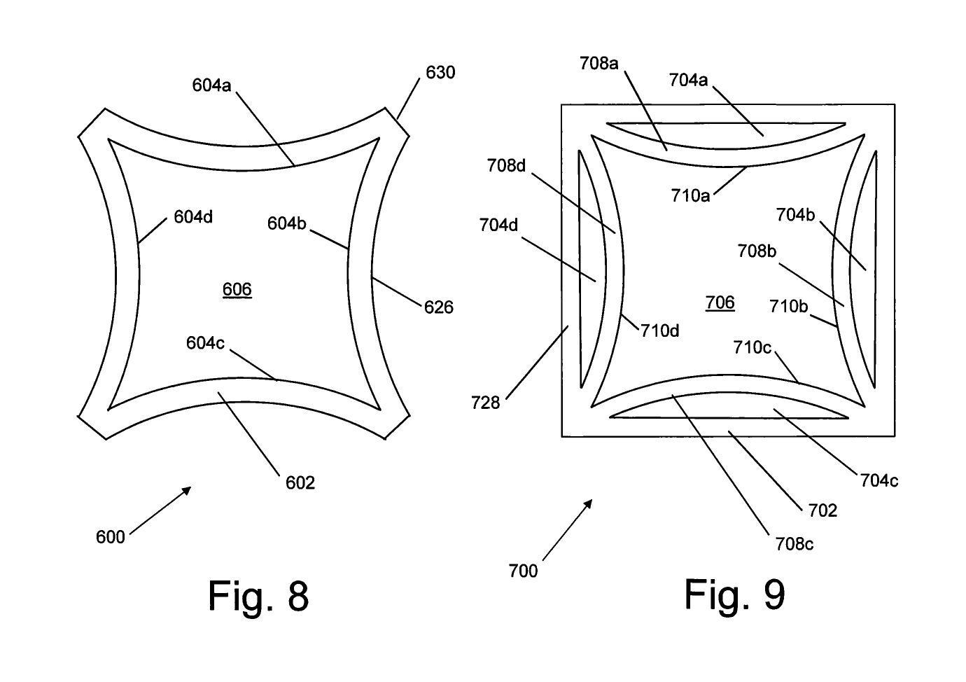

FIG. 8 illustrates an alternative embodiment of a connector 600. In this embodiment, the housing 602 is formed from a conductive material as described above and has a cross-section which has a center opening 606 having four convex interior surfaces 604a to 604d. The sides of the housing are thin enough to allow some flexing when an electrode is inserted in the center opening 606. When a connector electrode (not shown) from a lead is inserted into the center opening 606, the side walls of the housing 602 will yield, but be biased towards its manufactured shape. This yielding creates a pressure on the connector electrode to form a tight connection between an exterior surface of the connector electrode and the convex interior surfaces 604a-604d. A feedthrough wire 136 may be welded to an exterior surface 626 or to one or more of the corner faces 630 of the housing 602. Thus, an electrical connection can be established from the feedthrough wire (not shown) to a connector electrode (not shown) via the housing 602 without the use of additional springs, such as springs 204a and 204b of FIG. 4a.

FIG. 9 illustrates an alternative embodiment of a connector 700. A housing 702 is formed from a rectangular block of conductive material (as described above). In this embodiment, a center opening 706 and four side openings 704a-704d are created through the block to form four curved elements 708a-708d having convex surfaces 710a-710d. The curved elements 708a-708d of the housing 702 are thin enough to allow some flexing when an electrode (not shown) is inserted into the center opening 706. Thus, when a connector electrode from a lead is inserted into the center opening 706, the curved elements 708a-708d of the housing 702 will yield under pressure, but be biased towards their manufactured shape. This yielding creates a force on the connector electrode from the convex surfaces 710a-710d and will form a tight connection between an exterior surface of the connector electrode and the convex surfaces. A feedthrough wire (not shown) may be welded to an exterior surface 728. Thus, an electrical connection can be established from the feedthrough wire to a connector electrode via the housing 702 without the use of additional springs, such as springs 204a and 204b of FIG. 4a.

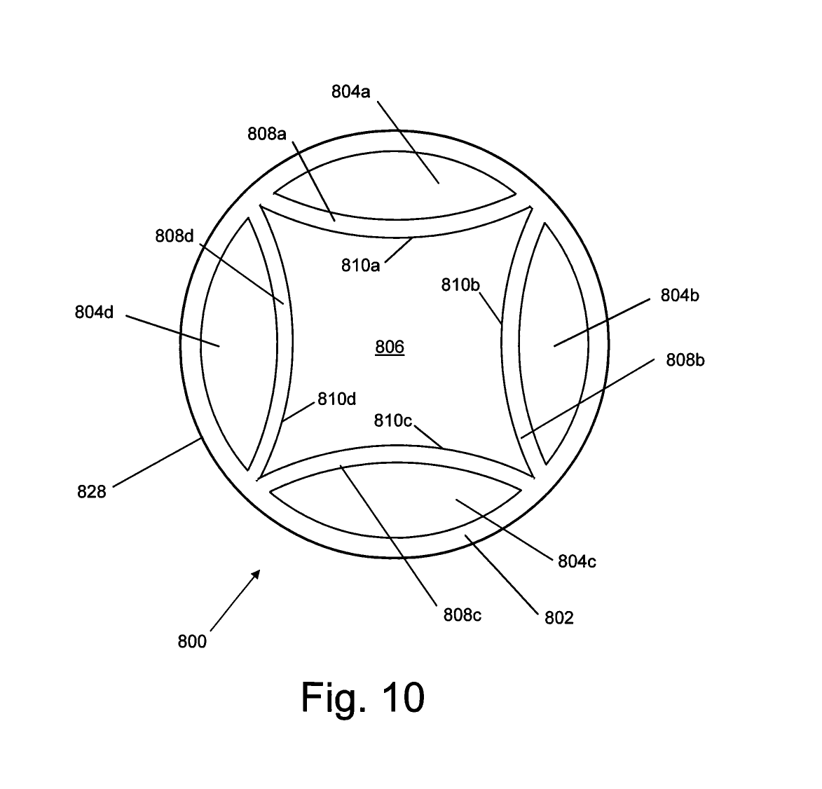

FIG. 10 illustrates an alternative embodiment of a connector 800. The housing 802 may be formed from a circular bar of conductive material (as described above). In this embodiment, a center opening 806 and four side openings 804a-804d are created through the bar. Therefore, the housing 802 has a cross-section having four curved side elements 808a-808d formed within the circular bar. In alternative embodiments, the side elements 808a-808d may be straight or nearly straight elements. The side elements 808a-808d of the housing are relatively flexible and allow some flexing when an electrode (not shown) is inserted into the center opening 806. Thus, when a connector electrode from a lead is inserted into the center opening 806, the side elements 808a-808b of the housing 802 will yield, but be biased towards their manufactured shape. This yielding creates a pressure on the exterior surface of connector electrode and will form a tight connection between the connector electrode and convex surfaces 810a-810d of the side elements 808a-808d, respectively. A feedthrough wire (not shown) may be welded to an exterior surface 828. Thus, an electrical connection can be established from the feedthrough wire to a connector electrode via the housing 802 without the use of additional springs, such as springs 204a and 204b of FIG. 4a.

FIGS. 11a and 11b illustrate an alternative embodiment of a connector 900. FIG. 11 a is a section view through the longitudinal middle of the connector 900. FIG. 11b is a front view of the connector 900. The housing 902 is formed from a circular tube 903 of conductive material (as described above) coupled to two side plates 911a and 911b positioned longitudinally on each end of the tube. For instance, in one embodiment, the tube 903 may be welded to the side plate 911b, which may be a circular donut shape plate having a center opening 906. Relatively flexible side elements or spring elements 908a-908d may then be inserted into the tube and placed radially around the center of the tube (or about the longitudinal axis of the tube). They may be curved or straight, but when inserted into the tube, they will bow towards the center of the tube as shown in FIG. 11a to form convex surfces 910a to 910b.

The donut shaped circular plate 911a may then be welded to the opposing side of the tube 903 to longitudinally retain the side elements 908a-908d as illustrated in FIG. 11b. Thus, in certain embodiments the side elements 908a-908d are prevented from moving longitudinally by the side walls 911a and 911b. The side elements 908a-808d of the housing are thin enough to allow some flexing when an electrode (not shown) is inserted into the center opening 906 of the side walls 911a and 911b.

In alternative embodiments, rather than positioning the length of side elements “in-plane” radially around the center of the tube as illustrated in FIG. 11a, a multitude of side elements or spring strips may be placed longitudinally (i.e., “out of plane” and generally parallel to the center or longitudinal axis of the tube 903) between the side walls 911a and 911b. The side walls 911a and 911b may then be pressed down and coupled to the tube 903. The pressing of the side wall 911a and side wall 911b into position will cause the longitudinally positioned strips to bend inwardly towards the center of the tube. Once the side walls 911a and 911b are coupled to the tube 903, the longitudinal springs would thus be maintained by the side walls 911a and 911b. The longitudinal springs would bow farther down than the side walls 911a and 911b so that they could engage the exterior surface of an electrode and thus make electrical contact.

Thus, regardless of whether the springs or side elements are placed longitudinally or radially (as illustrated in FIG. 11a and 11b) when a connector electrode from a lead is inserted into the center opening 906, the side elements 908a-808b will yield, but be biased because towards their manufactured shape. This yielding creates a pressure on the connector electrode and will form a tight connection between an exterior surface of the connector electrode and the convex surfaces 910a-910d of the curved side elements 908a-908d. A feedthrough wire (not shown) may be welded to an exterior surface 928 of the circular tube 903. Thus, an electrical connection can be established from the feedthrough wire to a connector electrode via the spring elements 908a-908d and the circular tube 903. Although four spring elements 908a-908b are illustrated in this embodiment, the scope of this present invention encompasses any number of spring elements.

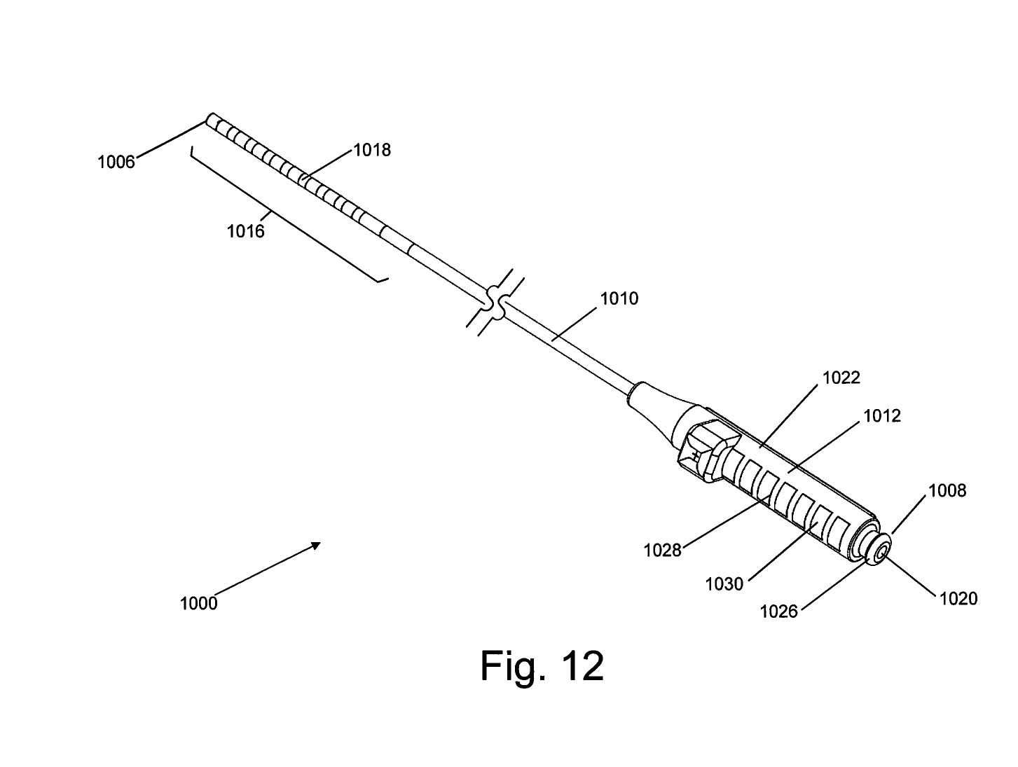

The various embodiments of the electrical connectors described herein may be used with a wide variety of medical treatment systems, such as neurostimulation systems. For instance, the various embodiments of the electrical connectors described above could also be used in a lead extension 1000 which is illustrated in FIG. 12.

FIG. 12 is a partial isometric illustration of a portion of the lead extension 1000. The lead 1000 is cut in length by break lines so that details of the lead are visible. Using the lead extension 1000 allows for a greater distance between the IPG 102 (not shown) and the stimulation electrode region 112 (FIG. 2) by physically extending the electrical connection between the IPG and the stimulation electrodes.

The lead extension 1000 includes a proximal end 1006 and a distal end 1008. Adjacent to proximal end 1006 of lead extension 1000 is a male connector region 1016 that comprises a plurality of connector or terminal electrodes 1018 which are sized to couple with the pulse generator 102 (as illustrated in FIG. 1) in a manner similar to that described above in reference to the leads 104a and 104b. Adjacent to the distal end 1008 is a female connector 1012 which is designed to receive the proximal connector region of a lead (such as the connector region 116 of a lead 104a as illustrated in FIG. 2).

The lead extension 1000 further comprises a flexible lead body 1010 that extends from proximal end 1006 to the female connector 1012. In certain embodiments, the lead body 1010 may have a structure, shape and material similar to the embodiments of the lead body 110 discussed above with reference to FIG. 2.

In certain embodiments, the plurality of connector electrodes 1018 may be formed of a conductive material similar to the connector electrodes 118. In a manner similar to that which is described for leads 104a and 104b in reference to FIG. 2, one or more conductors (not shown) extend along a substantial portion of the lead body 1010 to electrically connect the connector or terminal electrodes 1018 to corresponding electrical connectors (not shown) positioned within a connector housing 1022 of the connector 1012.

In certain embodiments, a connector housing 1022 is formed from materials are similar to the materials forming the header housing 122 described above. A receptacle 1020 receives the proximal end of a lead (not shown) and may incorporate a strain relief element 1026 to assist in protecting the lead in a manner similar to the strain relief elements 126a and 126b described above.

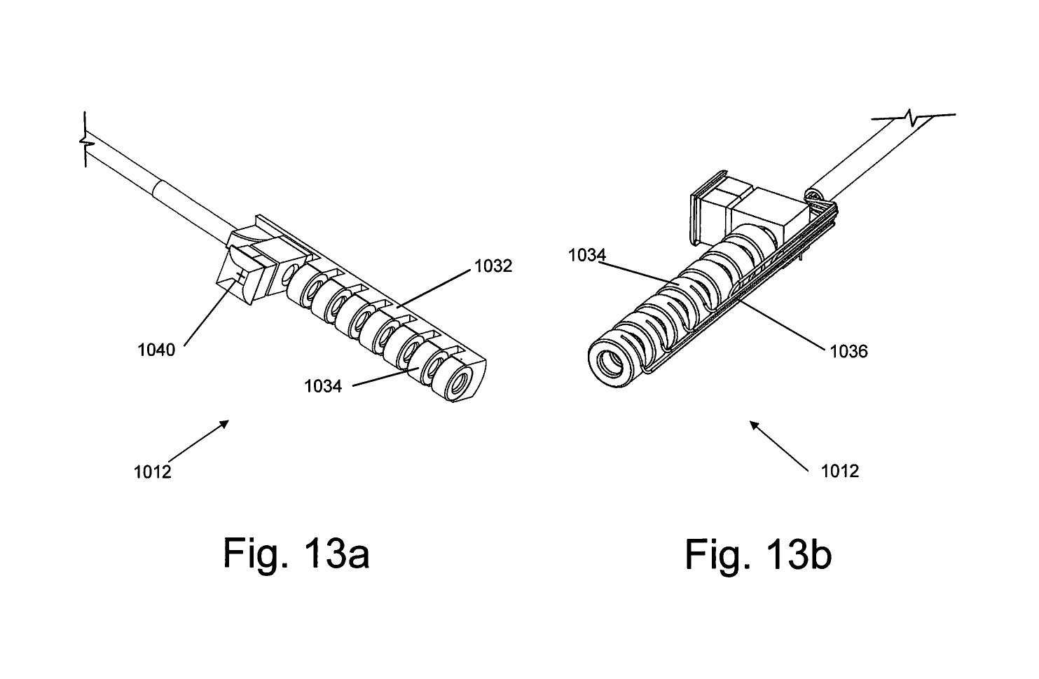

Defined within side walls of the connector housing 1022 is a plurality of recesses 1028 for housing a plurality of fillers 1030. In certain embodiments, a longitudinal filler element 1032 (FIG. 13a) is housed in a recess (not shown) on the opposing side of the connector housing 1022. FIG. 13a is an isometric view of the connector 1012 with the connector housing 1022 removed for clarity. In this figure, the longitudinal element 1032 is visible.

Refer now to both FIGS. 12 and 13a. The combination of the plurality of fillers 1030 and longitudinal filler element 1032 position and hold a plurality of electrical connectors 1034 housed within the connector housing 1022. The plurality of electrical connectors may be any of the electrical connectors described above with reference to FIGS. 4a through FIG. 11. The fillers 1030 and the longitudinal filler element 1032 are made from a compliant material. Once the fillers 1030 and the longitudinal filler element 1032 are assembled and positioned within the connector housing 1022, the compliant material characteristic of the fillers holds electrical connectors (not shown) in place by applying an elastomeric force to the electrical connectors. Additionally, when the connector 1012 is fully assembled and a stimulation lead is placed in the connector 1012 through the strain relief element 1026, the various conductive elements are sealed within the components of the connector housing 1022. Specifically, when extension lead 1000 is implanted within a patient, the housings and the strain relief element are designed to keep the electrical components sealed from contacting bodily fluids.

FIG. 13b is an isometric view of the female connector 1012 from an opposing angle to FIG. 13a where the connector housing 1022 and the longitudinal filler element 1032 have been removed for clarity. The plurality of electrical connectors 1034 are longitudinally spaced apart to match the longitudinal spacing of the connecting electrodes of the lead to be inserted into the connector 1012 so that when the ends of the lead are inserted into the plurality of electrical connectors 1034, the electrodes will be in contact with the electrical connectors.

A plurality of conductors 1036 extend from the lead extension body into the connector housing 1022 and are coupled to the plurality of electrical connectors 1034. In certain embodiments, the conductors 1036 are welded or soldered to an exterior surface of each connector in the plurality of connectors 1034 in a manner described above with regard to the feedthrough wires. Thus, an electrical connection can be established between the electrical connectors 1034 and the plurality of terminal electrodes 1018 via the electrical conductors 1036.

In certain embodiments, there may also be a “dummy” electrode (not shown) positioned at the distal end portion of the connection region 116 of the lead 104a or 104b as described above in relation to the header 121. A set screw (not shown) may be used to secure the dummy electrode to the female connector 1012. A self-sealing access port 1040 may thus be provided to allow for access to the set screw (not shown).

The IPG 102 and the lead extension 1000 are just two examples of implantable medical devices which could use the electrical connectors described in reference to FIGS. 4a through FIG. 11. Other embodiments of such electrical connectors may be used in a variety of implantable medical devices, such as systems for cardiac stimulation, peripheral nerve stimulation, deep brain stimulation, and gastric applications.

Although representative embodiments and advantages have been described in detail, it should be understood that various changes, substitutions and alterations can be made herein without departing from the spirit and scope of the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure that processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

Any combination of the features discussed above are within the scope of certain embodiments of the present invention. Thus, a feature disclosed in reference to one embodiment may be combined with another embodiment. Furthermore, combinations of disclosed features and alternative features are within the scope of certain embodiments of the present invention.

The abstract of the disclosure is provided for the sole reason of complying with the rules requiring an abstract, which will allow a searcher to quickly ascertain the subject matter of the technical disclosure of any patent issued from this disclosure. It is submitted with the understanding that it will not be used to interpret or limit the scope or meaning of the claims.

What is claimed is:

1. An implantable medical system comprising:
a least one stimulation lead, the stimulation lead comprising:
a lead body having a proximal end and a distal end;
a plurality of ring electrodes longitudinally positioned at a longitudinal spacing along a first region of the lead body adjacent to the proximal end of the lead body;
a plurality of stimulation electrodes positioned adjacent to the distal end of the lead body;
a plurality of conductors electrically connecting the plurality of ring electrodes to the plurality of stimulation electrodes,
a stimulation source, the stimulation source including:
a stimulation source housing containing electrical circuitry;
a header connected to the housing, wherein the header includes a
a header housing,
a plurality of electrical connectors longitudinally positioned within the housing to match the longitudinal spacing of the plurality of ring electrodes of the at least one lead and adapted to receive the plurality of ring electrodes of the at least one lead,
a plurality of feedthrough wires extending through the housing and coupled to the plurality of electrical connectors and the electrical circuitry, wherein each electrical connector in the plurality of electrical connectors further comprises,
a housing including:
a first transverse face,
a second transverse face on an opposite side of the housing,
a longitudinal bore having a longitudinal axis and defined within the housing spanning from the first transverse face to the second transverse face and sized to freely receive a ring electrode of the electrical lead,
at least one electrically conductive member coupled to the housing, the conductive member having:
a curved portion longitudinally positioned within the bore having a convex surface facing the center of the bore,
a first arm and a second arm extending from the curved portion, each of the first and second arms having:
a transverse portion extending in a transverse direction relative to the longitudinal axis,
a longitudinal portion extending from the transverse portion in a direction generally parallel to the longitudinal axis,
wherein the curved portion is positioned within the longitudinal bore and the longitudinal portions of the first and second arms are positioned adjacent to an exterior surface of the housing.

2. The system of claim 1, wherein the curved portion of the electrically conductive member is adapted to engage an exterior surface of a ring electrode of the plurality of ring electrodes and yield to pressure asserted by the ring electrode when the ring electrode is positioned within the longitudinal bore.

3. The system of claim 1, further comprising a first notch defined in the first transverse face of the housing sized to accommodate a portion of the transverse portion of the first arm and a second notch defined in the second transverse face of the housing sized to accommodate a portion of the transverse portion of the second arm.

4. The system of claim 1, wherein the housing is formed from a generally rectangular block of material.

5. The system of claim 4, further comprising at least one chamfered exterior surface spanning in a longitudinal direction from the first transverse face to the second transverse face and the longitudinal portions of the electrically conductive member are positioned adjacent to the chamfered exterior surface.

6. The system of claim 1, further comprising a second electrically conductive member coupled to the housing, the second conductive member having:
a second curved portion longitudinally positioned within the longitudinal bore having a second convex surface facing the center of the longitudinal bore,
a third arm and a fourth arm extending from the second curved portion, each of the third and fourth arms having:
a second transverse portion extending in a transverse direction relative to the longitudinal axis,
a second longitudinal portion extending from the second transverse portion in a direction generally parallel to the longitudinal axis,
wherein the second curved portion is positioned within the longitudinal bore and the longitudinal portions of the third and fourth arms are positioned adjacent to an exterior surface of the housing.

7. The system of claim 6, further comprising a second chamfered exterior surface spanning in a longitudinal direction from the first transverse face to the second transverse face and the longitudinal portions of the third and fourth arms of the second electrically conductive member are positioned adjacent to the chamfered exterior surface.

8. The system of claim 1 further comprising:
a lead extension, the lead extension comprising
a lead extension body having a proximal end and a distal end,
a plurality of extension ring electrodes longitudinally positioned at a longitudinal spacing along a first region of the lead extension body adjacent to the proximal end of the lead body,
a connector for receiving a the first region of the lead extension body, the connector including a second plurality of electrical connectors,
a plurality of extension conductors positioned within the lead extension body electrically connecting the plurality of extension ring electrodes to a second plurality of electrical connectors.

9. A electrical connector for implantable medical electrical devices, the connector comprising:
a housing including:
a first transverse face,
a second transverse face on an opposite side of the housing,
a longitudinal bore having a longitudinal axis and defined within the housing spanning from the first transverse face to the second transverse face and sized to freely receive an cylindrical electrode of an electrical lead,
at least one chamfered side surface spanning in a longitudinal direction from the first transverse to the second transverse face,
a first notch defined with the first transverse face and the chamfered side wall,
a second notch defined within the second transverse face and the chamfered side wall,
at least one electrically conductive strip member having:
a curved portion positioned within the longitudinal bore having a convex surface facing the center of the longitudinal bore,
two arms extending from the curved portion, each of the two arms having:
a transverse portion extending in a transverse direction relative to the longitudinal axis,
a longitudinal portion extending longitudinally from the transverse portion and over the curved portion,
wherein the curved portion is positioned within the longitudinal bore and the transverse portion of the first arm is partially positioned within the first notch and the transverse portion of the second arm is partially positioned within the second notch.

10. The electrical connector of claim 9, wherein a thickness of the curved portion of the at least one electrically conductive strip member will allow the curved portion to yield to a circular ring positioned within the longitudinal bore.

11. The electrical connector of claim 9, wherein the housing is made from a conductive material.

12. The electrical connector of claim 9, wherein the housing is made from a non-conductive material.

13. The system of claim 9, further comprising a second electrically conductive strip coupled to the housing, the second conductive member having:
a second curved portion longitudinally positioned within the longitudinal bore having a second convex surface facing the center of the longitudinal bore,
a third arm and a fourth arm extending from the second curved portion, each of the third and fourth arms having:
a second transverse portion extending in a transverse direction relative to the longitudinal axis,
a second longitudinal portion extending from the second transverse portion over the second curved portion,
wherein the second curved portion is positioned within the longitudinal bore and the second longitudinal portions of the third and fourth arms are positioned adjacent to an exterior surface of the housing.

14. The system of claim 13, further comprising a second chamfered exterior surface spanning in a longitudinal direction from the first transverse face to the second transverse face and the second longitudinal portions of the third and fourth arms are positioned adjacent to the chamfered exterior surface.

15. A electrical connector for implantable medical electrical devices, the connector comprising:
a housing including:
a first transverse face,
a second transverse face on an opposite side of the rectangular housing,
a longitudinal bore having a longitudinal axis and defined within the housing spanning from the first transverse face to the second transverse face and sized to freely receive an cylindrical electrode of an electrical lead, and
at least one electrically conductive strip member having a curved portion positioned within the longitudinal bore having a convex surface facing the center of the longitudinal bore.

16. The electrical connector of claim 15, wherein the conductive strip member is longitudinally positioned within the longitudinal bore.

17. The electrical connector of claim 15, wherein the conductive strip member is radially positioned within the longitudinal bore.

18. The electrical connector of claim 15, further comprising a first side wall coupled to the first transverse face and a second side wall coupled to the second transverse face.

19. The electrical connector of claim 15, wherein the housing is formed from a circular tube.

20. The electrical connector of claim 15, wherein the housing is formed from a rectangular tube.

21. The electrical connector of claim 15, wherein the conductive strip is integral with the housing.

22. The electrical connector of claim 15, further comprising a first notch defined on the first transverse face and a second notch defined on the second transverse face.

23. The electrical connector of claim 15, wherein the conductive strip further comprises a first arm and a second arm extending from the curved portion, each of the first and second arms having:
a transverse portion extending in a transverse direction relative to the longitudinal axis,
a longitudinal portion extending longitudinally from the transverse portion and over the curved portion,
wherein the curved portion is positioned within the longitudinal bore and the longitudinal portions are positioned adjacent to an exterior surface of the chamfered side wall.

24. The electrical connector of claim 23, wherein the transverse potion of the first arm is partially positioned within the first notch and the transverse portion of the second arm is partially positioned within the second notch.

25. The electrical connector of claim 15, wherein the at least one electrically conductive strip will yield to radially applied pressure.

26. The electrical connector of claim 15, wherein the housing is made from a conductive material.

27. The electrical connector of claim 15, wherein the housing is made from a non-conductive material.

28. A neurostimulation system kit comprising:
a pulse generator including a first series of electrical connections longitudinally spaced apart from each other at a predetermined distance,
at least one lead extension including:
a proximal end having a series of terminal electrodes sized and positioned to be received by the first series of electrical connections,
a distal end having a connector, the connector including a second series of electrical connections longitudinally spaced apart from each other at the predetermined position,
at least one medical lead, wherein a distal end portion of the medical lead includes series of stimulation electrodes and a proximal end portion includes a series of terminal electrodes spaced apart from each other at the predetermined distance wherein the terminal electrodes are sized and positioned to be received by either the first series of electrical connections or the second series of electrical connections,
wherein each electrical connector in the first or second series of electrical connectors includes:
a body,
a longitudinal opening defined within the body,
a conductive spring having a convex surface for engaging an exterior surface of an electrode, the spring positioned longitudinally within the longitudinal opening and having arms extending around at least one edge of the opening.

29. The neurostimulation kit of claim 28, wherein the body comprises at least one notch defined on the at least one edge and sized to receive a portion of the conductive strip.
